(19) United States
(12) Patent Application Publication
(10) Pub. No.: US 2024/0279573 A1
(43) Pub. Date: Aug. 22, 2024

(54) CHLORINE DIOXIDE CLEANER AND DISINFECTANT FOR USE ON FOOD CONTACT SURFACES

(71) Applicant: Spectrum Doxyicide Inc., Denver, CO (US)

(72) Inventor: Stephen Bradford Kong, Alamo, CA (US)

(73) Assignee: Spectrum Doxyicide Inc, Denver, CA (US)

(21) Appl. No.: 18/651,681

(22) Filed: Apr. 30, 2024

Related U.S. Application Data
(63) Continuation-in-part of application No. 17/143,141, filed on Jan. 6, 2021, now Pat. No. 11,970,675.
Application No. 17/143,141 is a continuation-in-part of application No. 16/995,804, filed on Aug. 17, 2020, now Pat. No. 11,497,215.
Application No. 16/995,804 is a continuation-in-part of application No. 16/884,033, filed on May 26, 2020, now Pat. No. 11,523,608.
Application No. 16/884,033 is a continuation-in-part of application No. 15/997,660, filed on Jun. 4, 2018, now Pat. No. 10,660,339.

(51) Int. Cl. C11D 3/48; A01N 25/30; A01N 59/08; C01B 11/02; C11D 1/12; C11D 3/04; C11D 3/395

(52) U.S. Cl. CPC C11D 3/48; A01N 25/30; A01N 59/08; C01B 11/024; C11D 1/123; C11D 3/044; C11D 3/3953

(57) ABSTRACT

Disclosed are devices, systems, and methods for producing a stabilized chlorine dioxide/surfactant solution based food contact cleaner and disinfectant product approved for use on food contact surfaces.

### CROSS-REFERENCE TO RELATED APPLICATIONS

This is a continuation-in-part of U.S. patent application Ser. No. 17/143,141 titled DEVICES, SYSTEMS, AND METHODS OF MAKING AND USING CHLORINE DIOXIDE BASED FORMULATION WITH IMPROVED STABILITY, filed Jan. 6, 2021, which is a continuation-in-part, to U.S. patent application Ser. No. 16/995,804 titled DEVICES, SYSTEMS, AND METHODS OF MAKING AND USING CHLORINE DIOXIDE BASED FORMULATION WITH IMPROVED STABILITY, filed Aug. 17, 2020, which is a continuation-in-part to U.S. patent application Ser. No. 16/884,033 titled DEVICES, SYSTEMS, AND METHODS OF MAKING AND USING CHLORINE DIOXIDE BASED FORMULATION WITH IMPROVED STABILITY, filed on May 26, 2020, which is a continuation-in-part to U.S. patent application Ser. No. 15/997,660 titled CHLORINE DIOXIDE BASED FORMULATION WITH IMPROVED STABILITY, filed on Jun. 4, 2018.

This application is also related to commonly owned U.S. application Ser. No. 14/631,806 titled BROAD SPECTRUM DISINFECTANT, filed on Feb. 25, 2015, which claims priority to U.S. Provisional Application No. 61/945,054, filed Feb. 26, 2014, which are all incorporated herein by reference.

### FIELD

The present invention is generally related to a broad spectrum cleaner and disinfectant using chlorine dioxide compositions, and in particularly, to chlorine dioxide combined with a surfactant approved for use on food contact surfaces.

### BACKGROUND

A sanitizer/disinfecting cleaning product for use on a food contact surface must provide a desired cleaner and disinfectant or sanitizer having suitable shelf-life stability. Disinfecting or sanitizing products require a suitable biocidal agent. Some examples of biocides are chlorine dioxide, hypochlorite, peroxide, and quaternary amines. The ingredients must also be approved by the U.S. Environmental Protection Agency (EPA) for use on food contact surfaces.

Many products containing reactive ingredients such a chlorine dioxide (ClO2) have problems with long-term stability, thus limiting their shelf-life. One particular advantage of chlorine dioxide over hypochlorite is that chlorine dioxide does not chlorinate organic compounds. However, it is inherently less stable than other biocides such as quaternary amines. Due to instability, most applications involve producing chlorine dioxide at the source of use. Therefore, chlorine dioxide must be properly formulated to be viable.

Chlorine dioxide is an effective biocide that can clean and deodorize. Chlorine dioxide has great potential for killing microorganisms. The presence of surfactants in a chlorine dioxide formulation facilitates cleaning of the surface, and improves germ kill. Many surfactants are not suitable for use in chlorine dioxide formulations because they promote the degradation of chlorine dioxide over time, thus decreasing the shelf-life of the product.

Chlorine dioxide in the absence of surfactant could be used on food contact surfaces. However, the elimination of surfactant would require a separate cleaning step prior to disinfecting/sanitizing. A one step cleaning and disinfecting product easier to use and preferred by consumers. The presence of an approved surfactant for food contact surfaces facilitates cleaning and disinfecting in one step.

While a chlorine dioxide based formula can be used to clean and disinfect surfaces, these formulations are not approved to be used on food contract surfaces. For a product to be approved for use on food contact surfaces, all the ingredients must be approved by the U.S. Environmental Protection Agency (EPA) for use on food contact surfaces.

In view of this, it is desirable to develop a chlorine dioxide food contact surface sanitizer/disinfecting cleaning product made with EPA approved ingredients for food contact surfaces. The creation of a food contact cleaner disinfectant fills a category of product that is currently not met by chlorine dioxide based cleaners. The chlorine dioxide based food contact cleaner should also maximizes the stability of the active ingredients so that the ClO2 based food contact cleaner has a suitable shelf-life and is ready to use after manufacturing.

### SUMMARY

Disclosed is sanitizer/disinfectant formula of stabilized chlorine dioxide (ClO2) made with a surfactant that is approved for food contact surfaces. The creation of stabilized chlorine dioxide/surfactant food contact cleaner disinfectant fills a category of consumer product that is currently not met by current chlorine dioxide based cleaners.

In one aspect, the invention is a stabilized chlorine dioxide/surfactant based food contact cleaner and disinfectant product approved for use on food contact surfaces. The stabilized chlorine dioxide/surfactant solution includes an approved sulfosuccinate anionic surfactant, such as, TRITON GR-5M surfactant, from Dow. The TRITON GR-5M surfactant is both compatible with chlorine dioxide and approved for use on food contact surfaces. The invention is not limited to TRITON GR-5M surfactant, other sulfosuccinate anionic surfactants approved for use on food contact surfaces may be used. Compatibility of the sulfosuccinate anionic surfactant with chlorine dioxide is critical to maximize the shelf life of the product. Using the sulfosuccinate type anionic surfactant achieves a suitable shelf life for the stabilized chlorine dioxide/surfactant solution. A number of synthesis reactions are known for producing chlorine dioxide.

In one embodiment, the stabilized chlorine dioxide/surfactant food contact cleaner may be produced in a batch process or a continuous process using Sodium dioxide (ClO₂), sulfosuccinate anionic surfactant, Sodium Hydroxide (NaOH) and Deionized water (H₂O).

Any suitable acid may be used in the process for producing ClO2. By suitable, we define a suitable acid to be one that reacts with NaClO2 to produce ClO2, and is compatible with ClO2. Acids may be moderate to strong acids that are capable of reacting with NaClO₂ to form ClO2. The process may be done using a single acid or a combination (mixture) of acids.

In another embodiment, the stabilized chlorine dioxide/surfactant food contact cleaner may be produced in a batch process or a continuous process making Sodium dioxide (ClO₂) using an acid solution mixed with Sodium chlorite (NaClO₂), sulfosuccinate anionic surfactant, Sodium Hydroxide (NaOH) and Deionized water (H₂O).

Surfactants help facilitate cleaning and wetting of surfaces to improve the micro efficacy of chlorine dioxide.

Gum thickeners can be added to thicken the product to improve contact time on a vertical surface or potentially as in a hand sanitizer.

The delivery of the stabilized ClO2/surfactant may be done in many different ways. In some embodiments, the delivery is a spray bottle and the stabilized ClO2/surfactant solution is a sprayable solution; the delivery is a wipe and the stabilized ClO2/surfactant solution is integrated into the wipe; the delivery is a tablet and the stabilized ClO2/surfactant solution is integrated into the tablet; the delivery is a laundry detergent with the stabilized ClO2/surfactant integrated into the laundry detergent; the delivery is a cleaning machine using stabilized ClO2/surfactant produced as a concentrate that can be used at full strength or diluted with water. In addition, a fragrance ingredient compatible with ClO2 may be added to produce a fragranced ClO2/surfactant solution.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through the use of the accompanying drawings.

### DETAILED DESCRIPTION

Embodiments of the invention will now be described with reference to the FIGURES, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The present invention is directed to producing a stabilized chlorine dioxide/surfactant solution using a surfactant the is approved for food contact surfaces creating a stabilized chlorine dioxide/surfactant solution with improved shelf-life. One approved surfactant is a sulfosuccinate anionic surfactant, such as TRITON GR-5M surfactant, from Dow. The inventor has found that the stability of the stabilized chlorine dioxide/surfactant solution can be significantly improved by the proper choice of pH, and through the careful choice of other product formula ingredients. By maximizing the stability of chlorine dioxide, the product has a suitable shelf-life and is ready to use after manufacturing. This stabilization benefit applies regardless of the reaction method used to produce chlorine dioxide.

There are limited chlorine dioxide formulations in the marketplace. The use of sulfosuccinate anionic surfactant with chlorine dioxide enables a sanitizer/disinfectant formula using chlorine dioxide that is approved for food contact surfaces. The creation of a food contact stabilized chlorine dioxide/surfactant cleaner disinfectant fills a category of consumer product that is currently not met by current chlorine dioxide based cleaners.

The inventor has found that improved stability of chlorine dioxide is due to adjusting the pH. This relationship between pH and stability of chlorine dioxide has not been recognized, and could explain the limited number of chlorine dioxide based product in the market. The increased stability of chlorine dioxide would make a product more desirable than a similar product with limited shelf-life, or a product that must be mixed prior to use.

Chlorine dioxide can be produced by a number of reactions. Several industrial methods of synthesis of chlorine dioxide are known such as acidification of chlorite, oxidation of chlorite by chlorine, oxidation of chlorite by persulfate. Other suitable reactions include the reaction of acetic anhydride with chlorite, the reduction of chlorates by acidification in the presence of oxalic acid, and the reduction of chlorates by sulfurous anhydride. Acidification of chlorite according to the following reaction is particularly appealing due to the availability, cost and ease of use of hydrochloric acid. It is understood that regardless of the method used to produce chlorine dioxide, the stability of the solution is controlled by pH and the proper choice of other ingredients.

It is envisioned that the stabilized chlorine dioxide/surfactant solution disclosed herein may be used on food contact surfaces. The present invention is not limited to food contact surfaces and can be used for many different applications and products, depending on the final dilution and concentration. For example, in some embodiments the stabilized chlorine dioxide/surfactant solution may be used in the laundry to sanitize, clean, deodorize or remove stains or destroy allergens on soft surfaces. In some embodiments the stabilized chlorine dioxide/surfactant solution may be used to clean, sanitize and/or disinfect floor, carpet, rug, drapes, bedding and furniture. In some embodiments the stabilized chlorine dioxide/surfactant solution may be used for stain removal and cleaning of floor, carpet, rug, furniture, drapes, bedding and other soft fabrics. In some embodiments the stabilized chlorine dioxide/surfactant solution may be used for odor control of floor, carpet, rug, furniture and drapes. In some embodiments the stabilized chlorine dioxide/surfactant solution may be used for disinfecting and sanitizing drapes, curtains, privacy screens, walls and floors and other materials and surfaces in hospitals. In some embodiments the stabilized chlorine dioxide/surfactant solution may be used against pathogens, mold and fungi in healthcare/medical facilities. In some embodiments the stabilized chlorine dioxide/surfactant solution may be used for cleaning, sanitizing and disinfecting soft toys, plastic toys, pacifiers, and other baby and childcare equipment, including but not limited to, high chairs, child car seats, push chairs and prams, swings, baby carriers, bikes, scooters, play pens. In some embodiments the stabilized chlorine dioxide/surfactant solution may be dispensed in aerosol devices to restrain or disinfect airborne bacteria to improve the indoor air quality.

The stabilized chlorine dioxide/surfactant solution may also be used for allergen control or allergen destruction. Allergens are proteins that cause an allergic response in our bodies. Runny noses and watery eyes are typical responses to allergens. Reducing the level of the allergen is needed to alleviate the allergic response. The stabilized chlorine dioxide/surfactant solution can clean, kill germs, deodorize and reduce allergens. Allergen destruction could be desirable in a carpet cleaner, floor cleaner, fabrics such as laundry or on furniture.

Other uses of the stabilized chlorine dioxide/surfactant solution include use as a sanitizer or disinfectant, floor cleaner, general cleaner/deodorizer, use in toilets, mouthwash or in laundry applications. The stabilized chlorine dioxide/surfactant solution may be used along or combined with other products.

In some embodiments the present invention is directed to improve the stability of stabilized chlorine dioxide/surfactant compositions and products by the proper choice of pH, and through the careful choice of other product formula ingredients. By maximizing the stability of chlorine dioxide, the stabilized chlorine dioxide/surfactant product has a suitable shelf-life and is ready to use after manufacturing.

In one embodiment, the stabilized chlorine dioxide/surfactant solution is produced using a method that includes combining a first amount of chlorine dioxide (ClO2) with a second amount of sulfosuccinate anionic surfactant and agitating the solution to distribute the sulfosuccinate anionic surfactant; adding a third amount of Sodium Hydroxide (NaOH) to adjust the pH of the resulting ClO2/surfactant solution to a desired pH. Additional water can be added to adjust the concentration of the resulting stabilized ClO2/surfactant solution.

In another embodiment, the stabilized chlorine dioxide/surfactant solution is produced using a method that includes adding a first amount of Hydrochloric acid (HCl) to a second amount of Sodium chlorite (NaClO2) that is dissolved in water and agitating the HCL and NaClO2 to mix the chemicals and allow the chemicals to react to completion producing chlorine dioxide (ClO2); adding a third amount of a sulfosuccinate anionic surfactant to the ClO2 and agitating the ClO2 and sulfosuccinate anionic surfactant mix to distribute the sulfosuccinate anionic surfactant to generate ClO2/surfactant; and after the reaction to generate ClO2/surfactant in solution has gone to completion, adding a fourth amount of Sodium Hydroxide (NaOH) to adjust the pH of the resulting ClO2/surfactant solution to a desired pH. Additional water or surfactant can be added to adjust the concentration of the ClO2/surfactant.

### Example 1

In some embodiments the invention is a method of making a stabilized chlorine dioxide/surfactant solution. The method includes adding 17.0 g/l 10% Hydrochloric acid (HCl) to 1.28 g/l Sodium chlorite (NaClO₂) dissolved in water; agitating the HCl and NaClO2 for at least 30 minutes to mix the chemicals forming chlorine dioxide (ClO2); adding 1.00 g/l of sulfosuccinate anionic surfactant and slowly agitate the ClO2 solution to distribute the sulfosuccinate anionic surfactant; and adding 36.2 g/l of 5% Sodium Hydroxide (NaOH) to adjust the pH of the ClO2 solution to a desired pH.

In some embodiments, the desired pH of the stabilized chlorine dioxide/surfactant solution is 2.3-8.2.

In some embodiments, the desired pH of the stabilized chlorine dioxide/surfactant solution is 3.6-7.5.

In some embodiments, the desired pH of the stabilized chlorine dioxide/surfactant solution is 4.0-6.5.

### Example 2

In some embodiments the invention is a method of making a high concentration stabilized chlorine dioxide/surfactant solution. The method includes adding 42.61 g/l 10% Hydrochloric acid (HCl) to 3.20 g/l Sodium chlorite (NaClO₂) dissolved in water; agitating the HCl and NaClO2 for at least 30 minutes to mix the chemicals forming chlorine dioxide (ClO2); adding 1.50 g/l of sulfosuccinate anionic surfactant and slowly agitate the ClO2 solution to distribute the sulfosuccinate anionic surfactant; and adding 42.23 g/l of 5% Sodium Hydroxide (NaOH) to adjust the pH of the ClO2 solution to a desired pH.

In some embodiments, the desired pH of the stabilized chlorine dioxide/surfactant solution is 2.3-8.2.

In some embodiments, the desired pH of the stabilized chlorine dioxide/surfactant solution is 3.6-7.5.

In some embodiments, the desired pH of the stabilized chlorine dioxide/surfactant solution is 4.0-6.5.

In some embodiments, the delivery of the stabilized chlorine dioxide/surfactant solution may be done in many different ways, for example, the delivery is a spray bottle and the stabilized chlorine dioxide/surfactant solution is a sprayable solution; the delivery is a wipe and the stabilized chlorine dioxide/surfactant solution is a solution integrated into the wipe; the delivery is a tablet and the stabilized chlorine dioxide/surfactant solution is integrated into the tablet; the delivery is a laundry detergent with the stabilized chlorine dioxide/surfactant solution is integrated into the laundry detergent; the delivery is a deodorizer with a fragrance ingredient that is compatible with the stabilized chlorine dioxide/surfactant solution; the delivery is a cleaning machine using stabilized chlorine dioxide/surfactant solution produced as a concentrate that can be used at full strength or diluted with water.

In some embodiments, the process may include a caustic, such as sodium hydroxide to adjust the solution pH. Depending on what acid and the amount of acid that is added to sodium chlorite, the corresponding amount of caustic (NaOH) needed to adjust the pH to stabilize the ClO2 will vary.
(Reference: Chlorine Dioxide by W. J. Masschelein, Ann Arbor Sciences 1979.)

### Product Applications/Uses

### Food Contact Surfaces

The disclosed stabilized chlorine dioxide/surfactant solution is an EPA/FDA approved cleaner and disinfectant for food contact surfaces. The stabilized chlorine dioxide/surfactant solution may be used full strength, or made in a concentrated solution can be diluted to clean and disinfect the food contact surfaces. The stabilized chlorine dioxide/surfactant solution could be used as a stand-alone floor cleaner product, or the stabilized chlorine dioxide/surfactant solution could be added to other cleaning formulas, provided that the stabilized chlorine dioxide/surfactant is compatible with the other cleaning formulas.

The food contact surfaces may include to tables, floors in butcher shops, slaughter houses, canneries, fruits/vegetable processing facilities, clean surfaces of the food prep equipment, etc. The stabilized chlorine dioxide/surfactant solution may be used to rinse/sanitize food containers, which could include bottles/containers for wine, beer, water, juices, soups, sauces, salad dressing, yogurt, distilled spirits, etc.

The stabilized chlorine dioxide/surfactant can be applied as a solution, applied using a spray bottle, or applied as a wet wipe, applied with a cleaning machine, or any other suitable methods of applying cleaners to food contact surfaces.

Some of the advantages of the stabilized chlorine dioxide/surfactant solution includes:
1) Dissipates/Dries Quicker—the stabilized chlorine dioxide/surfactant doesn't leave a residue since it basically reacts then dissipates into the air or evaporates with like water—it will dry at pretty much the same rate as any other water-based cleaner it just won't leave behind a residue like many other cleaners.
2) Odor Elimination—the stabilized chlorine dioxide/surfactant denatures proteins—breaks them down and breaks down other chemicals that are sensitive to powerful oxidation—almost anything that has an odor is prone to oxidation—therefore odors can be eliminated.
3) Effective Germ Kill—the stabilized chlorine dioxide/surfactant is among the most powerful oxidizers available and kills bacteria viruses, fungus, and other pathogens.
4) Environmentally Friendly—the stabilized chlorine dioxide/surfactant solutiuon does not leave a residue nor does it create chlorinated byproducts (a carcinogenic residue) like other germ kill products such as sodium hypochlorite.
5) Safe Around People and Pets—the stabilized chlorine dioxide/surfactant solution is food safe so you can feel confident the solution is okay for people and pets to be around.

### Other Applications/Uses

The stabilized chlorine dioxide/surfactant solution may be used at many different sites including: daycares, lavatories, restaurants, hotels, motels, salons, spas, gyms, offices, schools, homes, buses, automobiles, trains, planes, garages.

The stabilized chlorine dioxide/surfactant solution may be used on different surfaces including: glass, porcelain, laminate, ceramic tile, stainless steel, plastic, finished wood, metals, composite material.

The stabilized chlorine dioxide/surfactant solution may be used on many different objects including: kitchen, bathroom, home, counter tops, tables, desks, chairs, floors, exterior of appliances, refrigerator exterior, sinks, garbage cans/bins, diaper pails, water bottle exterior, high chairs, bathroom surfaces, toilet seats, toilet exteriors, light switches, door knobs, hand rails, telephones, computers, tools, shopping carts, gym equipment, kennels and cages, sink and counter, bathtub, faucets, shower areas, laptops, tablets, smart phones that are water resistant.

Below are examples of other product application and uses for the stabilized chlorine dioxide/surfactant solution.

### Laundry

The stabilized chlorine dioxide/surfactant solution may be used in the laundry to sanitize, clean, deodorize or remove stains or destroy allergens on clothes. It could be added through the wash, or added in the rinse cycle (i.e., post cleaning). High efficiency wash machines generally have chambers for various cleaning ingredients. Regular wash machines often have dispensers mounted on the agitator that dispense by centrifugal force to add a fabric softener during the rinse cycle. A dispensing ball or other device in the washing machine that can release the stabilized chlorine dioxide/surfactant solution after the wash step could be used. There could be injectors or other approaches for all the stabilized chlorine dioxide/surfactant solution to the wash or post-wash.

### Carpet/Rug Cleaning

The stabilized chlorine dioxide/surfactant solution may be used for floor and carpet/rug cleaning, sanitizing for pets. It could be a stand-alone spot cleaner or could be put in a carpet cleaning apparatus. The stabilized chlorine dioxide/surfactant solution could be incorporated into a cleaning product, or could be applied as a separate step after cleaning (post cleaning).

### Floor Cleaning

The stabilized chlorine dioxide/surfactant solution be used full strength, or a concentrated product can be diluted to clean floors. The stabilized chlorine dioxide/surfactant solution could be applied after cleaning the floor as a separate step i.e., post cleaning. It could be a stand-alone floor cleaner product or could be added to a cleaning formula provided that the ClO2 is compatible with the formula.

The stabilized chlorine dioxide/surfactant solution can be applied with a device such as a Swiffer™ (a device that dispenses a liquid as you mop). This device could also include the floor cleaner. Large floor cleaning machines are often seen to clean hallways in commercial buildings/hospitals etc.

### Hard Surfaces

The stabilized chlorine dioxide/surfactant solution can be at full strength or diluted for use on toilet bowl, sinks, cutting boards, highchairs, baby/kids toys, pacifiers, upholstery, kitchen ware. floors, laundry, etc.

### Decontamination of Clothing or Equipment

The stabilized chlorine dioxide/surfactant solution can be used for decontamination of hazmat suits or equipment. It could be used on farming equipment etc. to prevent transfer of bacteria/pathogens from one crop field to another.

### Fruit and Vegetable Wash

The stabilized chlorine dioxide/surfactant solution can be diluted and used as a wash for fruits and vegetables. Fruits and vegetables could be washed just as they are harvested, or could be washed in the consumer's home before eaten. Reducing molds could reduce spoilage and extend the storage of fruits and veggies.

The stabilized chlorine dioxide/surfactant solution can be used in agriculture and other plants: especially crops, e.g., sugar cane, rice, with fungal, viral and bacterial diseases;

### Personal Care for Humans

The stabilized chlorine dioxide/surfactant solution can be be used on “human surfaces” to control bacterial, viral and fungal diseases or contamination, e.g., mouthwash, gargle, rinse or other for throat, nose, douches skin, wounds or other internal uses.

The stabilized chlorine dioxide/surfactant solution can be either a concentrate or ready to use product could be used in applications such as; mouth wash, body wipe, wound care, treatment for skin infections or acne, and a potential remedy for poison oak. The concentration of ClO2 would vary depending on the application. We could combine ClO2 on a wipe substrate to be used on skin, as well as, on hard surfaces.

### Decontamination

The stabilized chlorine dioxide/surfactant solution can be used for decontamination before transportation (use as foot wash for shoes, boots, equipment, tires, etc.) when travelling between countries or sensitive agricultural or wildlife areas; e.g., prevent the spread of fungal or other contamination to the sensitive frog populations of the world by decontamination of the boots and equipment of the biologists studying the populations at various locations.

### Animals

The stabilized chlorine dioxide/surfactant solution can be used with animals both domesticated and wild to prevent fungal, viral and bacterial diseases.

The stabilized chlorine dioxide/surfactant solution can be used for animal wounds and skin disease treatments as well as hoof wash and other treatments including decontamination. Cleaning animal stalls could help prevent fungal, viral, and bacterial infections.

### Swimming Pools

The stabilized chlorine dioxide/surfactant solution can have applications in swimming pools and spas where ClO2 could be an alternative to sodium hypochlorite or sodium hypobromite.

### Mold/Mildew

The stabilized chlorine dioxide/surfactant solution can be kills mold and the mold can be decolorized by the stabilized chlorine dioxide/surfactant solution can be. Hence, you can kill mold and remove the dark mold stain with the same product. Some agents such as alcohol and high levels of quaternary ammonium compounds can/will kill mold but do not decolorize it.

### Teeth Whitening

As previously discussed, the stabilized chlorine dioxide/surfactant solution can be used as a mouth wash/oral rinse etc. In addition, the stabilized chlorine dioxide/surfactant solution can be could possibly be used to whiten teeth.

### Germs/Allergens

The stabilized chlorine dioxide/surfactant solution can be used to kill germs and destroy allergens on hard or soft surfaces for household, commercial and healthcare (including equipment) with bacterial, viral or fungal diseases or contamination.

### ClO2 Components

### Acids

Any suitable acid may be used in the process disclosed. We define suitable as an acid that reacts with NaClO2 to form ClO2 and is also compatible with ClO2. Such acids generally have a pKa less than about 5. For example, but not limited to, hydrochloric acid, sulfuric acid, nitric acid, perchloric acid, malonic acid, citric acid, sulfamic acid, succinic acid, and oxalic acid.

Acids may be moderate to strong acids that are capable of reacting with sodium chlorite to form ClO2. The strongest acids are “mineral acids” which completely dissociate in aqueous solution. Common examples of strong acids are hydrochloric acid, sulfuric acid, and nitric acid. These are characterized as having pKa s<1. Strong acids react very quickly with sodium chlorite.

Moderately strong acids include many organic acids. Examples include, citric acid, sulfamic acid, succinic acid, and oxalic acid. These are characterized as having pKa values less than about 5. The acid needs to react with sodium chlorite to form ClO2. If the pKa is too high, the reaction will not occur or will be very slow. Generally, the lower the pKa, the stronger the acid and the faster the reaction with sodium chlorite.

### Combination of Acids

The reaction of NaClO2 with an acid is used to produce ClO2. The acid can be mineral acid or organic acids. Mineral acids have pKa<1. Organic acids have a pKa<5. Adding excess acid, which is the amount required by the stoichiometry of the reaction will speed up the reaction and maximize the conversion of chlorite to ClO2.

The process may be done using a combination (mixture) of acids rather than just using one acid.

In some embodiments, formulations without the addition of any ingredients other than the ClO2 that is produced may be desirable.

### Surfactants

Surfactants help facilitate cleaning and wetting of surfaces to improve the micro efficacy of chlorine dioxide. The key criteria are that the surfactants must be reasonably compatible with ClO2 and other potential formula ingredients, and must function in the particular product. The formulas presented herein use sulfosuccinate anionic surfactant, such as TRITON GR-5M, that is approved for food surface cleaning. The present invention is not limited to TRITON GR-5M for the surfactant.

The sulfosuccinate anionic surfactant can be added at any time during the production of ClO2. It can be added to already produced ClO2. It can be added after the ClO2 solution is formed is formed. It can during the production of ClO2. It can be added after the pH is adjusted.

### Other Adjuncts

Gum thickeners can be added to thicken the product to improve contact time on a vertical surface or potentially as in a hand sanitizer. For example, gum thickeners may include, but not limited to, xanthan gum, Kelzan AP-AS (from CP Kelco), Keltrol (from CP Kelco) or other suitable gum thickener. The key is that the adjuncts must be reasonably stable with chlorine dioxide. The benefits of proper pH choice also apply to the addition of adjuncts. We are not limited to just these two ingredients. Optimizing the pH will improve stability of formula containing the desired adjuncts.

### Other Components

Other formula components are possible including fragrances, dyes, enzymes, cleaning agents, anti-redeposition agents, wipe substrates, brighteners etc. Additional oxidant stable ingredients e.g., scents/fragrance ingredients, foaming or de-foaming ingredients, may also be added. The choice of these other components depends on the product application. A key feature is that they should be compatible with ClO2.

### Concentrated Product

A concentrated stabilized chlorine dioxide/surfactant solution could have applications such as a floor cleaner, general cleaner/deodorizer, use in toilet bowl, or in laundry applications. The concentrated stabilized chlorine dioxide/surfactant solution is a product that may be used full strength or be diluted prior to use. The stabilized chlorine dioxide/surfactant solution is prepared using a stabilized formula and then diluted by adding additional water or adding the concentrated formula to water. An example of a concentrated product and how one is used, is Pine-Sol™. Pine-Sol™ can be used full strength or diluted. The standard dilutions would apply. i.e., add product to an equal amount of water to the stabilized chlorine dioxide/surfactant solution would dilute it by 50% etc. The concentrate could be a diluted to refill for a spray product.

It would also be possible to make a two-part product where the acid and chlorite are separated until they are combined to react. The surfactant can be added to either product. Since there is no ClO2 produced until acid and chlorite react in a two-part product, the stability issues of ClO2 could be avoided. After mixing the acid and sodium chlorite and surfactant, the resulting stabilized chlorine dioxide/surfactant solution could be further stabilized by adjusting its pH.

The procedure to prepare a stabilized chlorine dioxide/surfactant solution concentrate is the same as discussed above. We would add excess acid to sodium chlorite and wait for the reaction to produce ClO2 to go to completion. Surfactant could be present either before or after the reaction of acid and chlorite. Then adjust the pH so that the product is in the pH in the range that provides improved stability. The concentrate is then ready for use.

The concentration of the chlorine dioxide and the surfactant would be higher in the concentrated product to allow for dilution so that the diluted product will still have a concentration to be effective. Higher concentration of ClO2 may be used to sanitize or disinfect while a lower concentration of ClO2 in the diluted form may be good for general cleaning and deodorizing.

The concentrated stabilized chlorine dioxide/surfactant solution should be safe to use and have a suitable shelf life for storage.

In some embodiments the stabilized chlorine dioxide/surfactant solution may include other ingredients, such as fragrance, dyes, or thickeners, etc. to change the aesthetics or the form of the product or even change other performance attributes. In some embodiments gum may he added to make a gel product. These other embodiments should be chosen based on compatibility with ClO2 for a suitable shelf life, as well as, for its intended function.

### Dilution-Preparation of Finished Product

Deionized water should be used to prepare or dilute the stabilized chlorine dioxide/surfactant solution during production of the finished product. The pH of the finished product should be adjusted to improve stability and/or to achieve the desired product pH. If the stabilized chlorine dioxide/surfactant solution is required to have a specific pH, the overall stability of the ClO2 could subsequently be affected. It is therefore preferred to have the product pH fall within the pH range that promotes the improved stability.

### ClO2 Product Delivery

It is envisioned that the stabilized chlorine dioxide/surfactant solution be delivered in many different forms using many different devices or applicators for delivery, depending on the application. Below are some non-limiting examples.

Food Contact Surfaces Sanitizer or Disinfectant—For food contact surfaces, the stabilized chlorine dioxide/surfactant solution may be packaged in a spray bottle or package of wipes. The stabilized chlorine dioxide/surfactant solution may be provided in a concentrated solution either at full concentration or diluted for use in a cleaning machine.

Sanitizer or Disinfectant—For hard surfaces, the stabilized chlorine dioxide/surfactant solution may be packaged in a spray bottle or package of wipes. For hand sanitizer, the stabilized chlorine dioxide/surfactant solution be in a squirt or pump bottle. For soft surfaces, the stabilized chlorine dioxide/surfactant solution may be provided in a concentrated solution either at full concentration or diluted that can be used/applied with a cleaning machine, such as a carpet cleaner, vacuum, floor cleaner, steam cleaner, or other cleaning/disinfection machine.

Floor Cleaner—The stabilized chlorine dioxide/surfactant solution may be provided in a concentrated solution that can be used either at full concentration or diluted, such as Pine-Sol™. The stabilized chlorine dioxide/surfactant solution may be applied using a cleaning machine, such as a hard floor cleaner or sweeper capable of delivering fluids to the floor.

General cleaner/deodorizer—The stabilized chlorine dioxide/surfactant solution may be provided in a spray bottle, like Lysol Bathroom Cleaner™, or disinfecting wipes like Clorox™ Wipes™.

Toilet—The stabilized chlorine dioxide/surfactant solution may be provided in a tablet form to drop-in the bowl or put in the toilet tank for each flush, like Clorox™ tablets. The tablets may be different concentrations, such as the drop-in bowl tablet may have a higher concentration than the tank tablet.

Laundry—The stabilized chlorine dioxide/surfactant solution may be mixed in a laundry detergent; or may be a separate solution additive, like Lysol™ Laundry Sanitizer Additive, that is added through the wash, or added in the rinse cycle; or as beads that are thrown in the wash, like Downy™ Fresh Scent Booster Beads; or as a ball or other device in the washing machine that could release the ClO2 after the wash step.

### General Instructions

All chemicals are used without further purifications. All sample bottles used were amber and appropriately labeled. Each container was rinsed with deionized water before reusing.

All processes and reactions are carried out at room temperature not exceeding (22° C.) unless otherwise specified.

The present invention may be used for various products, including, for example, a surface disinfectant or sanitizer. While the present application discloses embodiments for a surface disinfectant, it is contemplated that the same processes, methods, and solutions may be used for the other products.

### Production Process

The production/manufacturing for the stabilized chlorine dioxide/surfactant solution should follow general manufacturing guidelines that are typically followed in the production of hypochlorite or peroxide containing based products. All contact surfaces in the production equipment, filling and line and packaging should be in good condition. They must/should be emptied and thoroughly rinsed so as to prevent cross contamination prior to use. Such practices are generally followed in the production of hypochlorite containing products or other products where contamination is undesirable/not tolerated.

Preferably, the entire production process for the solution would be conducted under clean room conditions, in order to minimize the possibility of contamination of the solution by environmental contaminants, such as airborne particles. All contact surfaces, including without limitation surfaces of production equipment, filling equipment and packaging, should be thoroughly cleaned of contaminants prior to use.

### Example 1—Batch Process for Preparation of the Stabilized Chlorine Dioxide/Surfactant Solution Starting with Chlorine Dioxide

Ranges for the amounts of the Solutions to be used for each embodiment are shown above.
1. Prepare the mixing vessel by decontaminating the container with chlorine dioxide followed by a rinse with deionized water. If the container is used regularly, the container may be rinsed with only deionized water.
2. Add chlorine dioxide to the mixing vessel.
3. Add the sulfosuccinate anionic surfactant to the chlorine dioxide. Mix or slowly agitate to distribute the sulfosuccinate anionic surfactant in the chlorine dioxide.
6. Adjust the pH with chlorine dioxide/surfactant solution to achieve the target pH for a stable solution.
7. Store samples in sealed opaque/dark containers.
   In the procedure described above, it is also acceptable to add the surfactant after the pH is adjusted.

### Example 2—Batch Process for Preparation of the Stabilized Chlorine Dioxide/Surfactant Solution Producing the Chlorine Dioxide

1. Prepare the mixing vessel by decontaminating the container with chlorine dioxide followed by a rinse with deionized water. If the container is used regularly, the container may be rinsed with only deionized water.
2. Add deionized water corresponding to size of the batch followed by the sodium chlorite. Allow the sodium chlorite to completely dissolve. Agitate the sodium chlorite solution.
3. Add the hydrochloric acid to the sodium chlorite solution. After the hydrochloric acid is added, the vessel should be loosely capped to allow the release of any gas that may have formed in the container. The amount of gas formed will vary depending on the concentrations of hydrochloric acid and sodium chlorite present.
4. Allow the acid-chlorite mixture to react until completion with slow agitation forming chlorine dioxide.
5. Add the sulfosuccinate anionic surfactant to the chlorine dioxide. Mix or slowly agitate to distribute the sulfosuccinate anionic surfactant.
6. Adjust the pH with chlorine dioxide/surfactant solution to achieve the target pH for a stable solution.
7. Store samples in sealed opaque/dark containers.

### Example 3—Continuous Process Preparation of Solution Starting with Chlorine Dioxide

Below shows one embodiment of a continuous process starting with prepared chlorine dioxide.
A. Turn on the water pump in the reactor unit and adjust the deionized water to the desired feed rate.
B. Turn on the chemical solutions feed pumps and set the feed rates to the desired percentage of chlorine dioxide and sulfosuccinate anionic.
C. Assure proper mixing of the chlorine dioxide and sulfosuccinate anionic surfactant.
D. Adding sodium hydroxide to adjust the pH.
E. Assure proper mixing of the water and chemicals.

### Example 4—Continuous Process Preparation of Solution with Processing the Chlorine Dioxide

Below shows one embodiment of a continuous process for preparing chlorine dioxide Surface Disinfectant.
A. Turn on the water pump in the reactor unit and adjust the deionized water to the desired feed rate.
B. Turn on the chemical solutions feed pumps and set the feed rates to the desired percentage of hydrochloric acid, sodium chlorite and sulfosuccinate anionic surfactant.
C. Assure proper mixing of the hydrochloric acid, sodium chlorite and sulfosuccinate anionic surfactant, forming chlorine dioxide with surfactant.
D. Adding sodium hydroxide to adjust the pH.
C. Assure proper mixing of the water and chemicals.

It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claims set forth here below not be construed as being order-specific unless such order specificity is expressly stated in the claim.

The invention claimed is:

1. A method of making a stabilized solution of chlorine dioxide having improved long term stability for use on food contact surfaces comprising:
adding an amount of sulfosuccinate anionic surfactant to an amount of Chlorine Dioxide (ClO2); and
adding an amount of Sodium Hydroxide (NaOH) to adjust the pH of the resulting ClO2/surfactant solution to a target value;
where the pH is adjusted after the reaction to generate ClO2/surfactant in solution has gone to completion.

2. The method of claim 1, wherein the sulfosuccinate anionic surfactant is TRITON GR-5M surfactant.

3. The method of claim 1, wherein the pH target value is 2.3-8.2.

4. The method of claim 1, wherein the wherein the pH target value is 3.6-7.5.

5. The method of claim 1, wherein the wherein the pH target value is 4.0-6.5.

6. The method of claim 1, wherein the Chlorine Dioxide (ClO2) is made by:
adding an amount of Hydrochloric acid (HCl) to an amount of Sodium chlorite (NaClO2) that is dissolved in water forming the chlorine dioxide (ClO2).

7. The method of claim 6, wherein:
the amount of Hydrochloric acid (HCl)=17 g/l; 10% HCl;
the amount of Sodium chlorite=1.28 g/l NaClO2 (80%) dissolved in water;
the amount of sulfosuccinate anionic surfactant=1.0 g/l;
the amount of Sodium Hydroxide=36.2 g/l of 5% NaOH.

8. The method of claim 6, wherein:
the Hydrochloric acid (HCl)=42.61 g/l 10% HCl;
the amount of Sodium chlorite=3.20 g/l NaClO2 dissolved in water;
the amount of sulfosuccinate anionic surfactant=1.50 g/l;
the amount of Sodium Hydroxide=42.23 g/l of 5% NaOH.

9. A method for producing a stabilized solution of chlorine dioxide having improved long term stability for use on food contact surfaces comprising:
1) adding hydrochloric acid to sodium chlorite dissolved in water;
2) agitating the solution of hydrochloric acid and sodium chlorite until the reaction to form chlorine dioxide is complete;
3) adding sulfosuccinate anionic surfactant to the chlorine dioxide solution;
4) slowly agitating the chlorine dioxide and sulfosuccinate anionic surfactant solution to distribute the sulfosuccinate anionic surfactant; and
5) adding sodium hydroxide to the chlorine dioxide/surfactant solution to adjust the pH to a target value;
wherein:
the amount of hydrochloric acid=17.0 g/l 10% hydrochloric acid solution;
the amount of sodium chlorite=1.28 g/l NaClO2 (80%);
the amount of sulfosuccinate anionic surfactant=1.00 g/l; and
the amount of sodium hydroxide=36.2 g of 5% NaOH.

10. The method of claim 9, wherein the sulfosuccinate anionic surfactant is TRITON GR-5M surfactant.

11. The method of claim 9, wherein the desired pH range is 2.3-8.2.

12. The method of claim 9, wherein the desired pH range is 3.6-7.5.

13. The method of claim 9, wherein the desired pH range is 4-3.5.

14. A method for producing a stabilized solution of chlorine dioxide having improved long term stability for use on food contact surfaces comprising:
1) adding hydrochloric acid to sodium chlorite dissolved in water;
2) agitating the solution of hydrochloric acid and sodium chlorite until the reaction to form chlorine dioxide is complete;
3) adding sulfosuccinate anionic surfactant to the chlorine dioxide solution;
4) slowly agitating the chlorine dioxide and sulfosuccinate anionic surfactant solution to distribute the sulfosuccinate anionic surfactant; and
5) adding sodium hydroxide to the chlorine dioxide/surfactant solution to adjust the pH to a target value
wherein:
the amount of hydrochloric acid=42.61 g/l 10% HCl;
the amount of sodium chlorite=3.20 g/l NaClO2;
the amount of sulfosuccinate anionic surfactant=1.50 g/l; and
the amount of sodium hydroxide=42.23 g/l of 5% NaOH.

15. The method of claim 14, wherein the sulfosuccinate anionic surfactant is TRITON GR-5M surfactant.

16. The method of claim 14, wherein the desired pH range is 2.3-8.2.

17. The method of claim 14, wherein the desired pH range is 3.6-7.5.

18. The method of claim 14, wherein the desired pH range is 4-3.5.
